(19) United States
(12) Patent Application Publication
(10) Pub. No.: US 2022/0409450 A1
(43) Pub. Date: Dec. 29, 2022

(54) SYSTEM AND METHOD FOR STOCKING WORKPLACE FIRST AID CABINETS

(71) Applicant: Cintas Corporate Services, Inc., Cincinnati, OH (US)

(72) Inventors: Cara Beaumont, Lebanon, OH (US); Lindsey DeLoye, Flat Rock, MI (US); Todd VanHouten, Rochester, NY (US)

(21) Appl. No.: 17/844,298

(22) Filed: Jun. 20, 2022

Related U.S. Application Data
(60) Provisional application No. 63/213,779, filed on Jun. 23, 2021.

(51) Int. Cl. A61F 17/00; B65D 5/48; B65D 5/54

(52) U.S. Cl. CPC A61F 17/00; B65D 5/48014; B65D 5/542; A47B 67/02

(57) ABSTRACT

A system and a method of stocking workplace first aid cabinets includes a variety of first aid items such as adhesive bandages, fabric bandages, comfort bandages, tapes, wraps and other items which are varied to match a user's skin tone. In one aspect, a box or container holds a number of similar items which may only vary one from another based on their color to match one's skin tone. Discrete compartments are provided in the box, each of which may hold a number of items, such as bandages or like, of similar skin tone color such that the entire contents of the box is a number of similar items arranged into discrete compartments based on their color relative to one's skin tone. This invention may be sold and serviced to a workplace in connection with vehicle delivered, business to business first aid cabinet stocking and re-stocking services.

This claims the benefit of U.S. Provisional Patent Application Ser. No. 63/213,779, filed Jun. 23, 2021 and hereby incorporated by reference in its entirety.

### BACKGROUND OF THE INVENTION

This invention generally relates to systems and methods for stocking workplace first aid cabinets, and more particularly, to a container for a variety of similar first aid or safety items useful in the stocking of such cabinets.

Annually, over 3.6 million nonfatal occupational injuries and illnesses are reported for private and public sector workers. State and federal regulations often require employers to ensure that safety and first aid supplies are readily available for employees at the workplace. Occupational Safety and Health Administration (OSHA) has been established to ensure safe and healthful working conditions for working men and women by setting and enforcing standards and by providing training, outreach, education and assistance. OSHA and other regulations mandate that adequate first aid supplies are readily available in the private and public workplaces.

Such supplies are commonly provided in one or more cabinets conveniently accessible to the workers at the workplace. Employers and workplaces which outsource their first aid cabinet service are nearly twice as likely to have confidence that their cabinets and supplies meet or exceed applicable American National Standards Institute (ANSI) standards. Readily accessible and appropriately stocked safety and first aid cabinets are an important part of a safe and product workplace. Occupational illnesses and injuries cost businesses over $170 billion yearly in the US.

First aid cabinets which are easy to use and stocked with effective, appropriate and quality supplies are important in the workplace. Service providers for the installation and stocking of such first aid cabinets, such as the assignee of this invention, Cintas Corporation, are often an integral component of the safety and well-being of a workplace's employees. Customization and maintenance of the first aid cabinet and its contents is an important factor in workplace safety. Unfortunately, too many workplaces fail to properly install and/or maintain their first aid cabinets for the particular risks associated with the relevant workplace and the demographics of their workforce. Even if the cabinet is initially installed and stocked appropriately, too many businesses fail to maintain the cabinets over time by not re-stocking, replenishing and/or updating the contents of the cabinet for the relevant workforce. Too often the contents of the cabinet become disorganized and/or depleted such that the relevant safety standards and regulations are not met and the cabinet proves to be inadequate when the safety or first aid of an employee is at issue.

It is well known in the art to provide a bandage or other first aid item to match a person's skin tone, as exemplified by the contents of U.S. Pat. No. 6,297,420, the contents of which are hereby incorporated by reference in its entirety. Workplace first aid cabinets by their very nature are intended to service each of the individuals in the workplace and their unique needs and desires. Stocking the cabinet with bandages applicable to each of the members of a workplace staff can become very cumbersome and demanding from an inventory perspective. Combining such demands with the all too common inattention to the stocking and replenishing of the workplace cabinet significantly complicates the situation. Such complications and issues may unfortunately be realized when the contents of the cabinet may be needed in an emergent or critical care situation when time may be of the essence and quick and convenient access to the cabinet supplies is needed.

Therefore, a solution to these and other problems in the installation, stocking and maintenance of first aid cabinets for the relevant workforce is needed.

### SUMMARY OF THE INVENTION

This invention addresses these and other shortcomings in the prior art. In various embodiments, this invention is directed to a system and a method of stocking workplace first aid cabinets including a variety of first aid items such as adhesive bandages, fabric bandages, comfort bandages, tapes, wraps and other items.

In various embodiments, this invention includes a system for the stocking, arrangement and servicing of a workplace first aid cabinet. In another aspect, this invention includes a method for stocking, arranging and servicing the workplace first aid cabinet. In a further aspect, this invention is directed toward a system and an associated method for stocking, servicing and replenishing particular items in the workplace first aid which are varied to match a user's or patient's or employee's skin tone.

In a further aspect, this invention includes a box or container for holding a number of similar items which may only vary one from another based on their color to match one's skin tone. More specifically, in one aspect this invention includes a box or container with discrete compartments each of which may hold a number of items, such as bandages or like, of similar skin tone color such that the entire contents of the box or container is a number of similar items arranged into discrete compartments based on their color relative to one's skin tone.

This invention may be sold and serviced to a workplace in connection with vehicle delivered, business to business first aid cabinet stocking and re-stocking services. The initial delivery could utilize a service sales representative (SSR) placing a box according to this invention into first aid cabinet located at a customer's workplace. The box may include a removable top which the SSR will remove and place the stocked box in the cabinet on a shelf near other bandage supplies. During ongoing subsequent service for the workplace establishment, the SSR will either refill the compartments that are low in inventory which may be specific to a particular skin tone or replace the entire box if sufficient inventory is depleted. To facilitate the refill process, each SSR may carry inventory of “refill packs” for each skin tone color item or bandage, for the express purpose of restocking these boxes.

It is believed that there is no such boxed product for first aid cabinets or associated service in the prior art that includes multiple compartments to organize similar but distinct products, which in one embodiment may be distinguished by a user's skin tone. Additional skin tone products, including bandages may include other sizes or shapes such as knuckle bandage, large patch bandage and the like. This invention is not limited to bandages but may include any product that is available in similar variations but has at least one distinction which may be contained in discrete portions of one shared box. The unique features of this invention include the combining of multiple products/colors/shapes into one box and separating them into compartments. In addition to the compartments making it easier to service/restock, it also will make it easier for the individual user (employee) to quickly find and take the product they need.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a view of a service sales representative (SSR) servicing a first aid cabinet in a workplace setting according to one embodiment of this invention;

FIG. 2 is a view of one portion of the cabinet of FIG. 1 showing various items in the cabinet;

FIG. 3 is a view of another portion of the cabinet of FIG. 2 showing various items in the cabinet, including boxes of bandages according to one embodiment of this invention;

FIG. 4 is a plan view of exemplary bandages of a variety of shapes which may be contained in the box according to one aspect of this invention;

FIG. 5 is a plan view of further exemplary bandages of a variety of shapes which may be contained in the box according to one aspect of this invention;

FIG. 6 is a perspective view of further exemplary products which may be contained in the cabinet according to one aspect of this invention;

FIG. 7 is a perspective view of further exemplary products which may be contained in the cabinet according to one aspect of this invention;

FIG. 8 is a flow chart of another aspect of servicing the first aid cabinet according to another aspect of this invention;

FIG. 9 is a view of two users of differing skin tone each bearing a bandage matching their skin tone;

FIG. 10 is perspective view of the carton of FIG. 10 showing the discrete compartments within the carton;

FIG. 11 is a perspective view of an erected carton or box according to one aspect of this invention;

FIG. 12 is a perspective view of the carton of FIGS. 10-11 with a top portion of the carton being removed;

FIG. 13 is a perspective view of the carton with each compartment containing items, such as bandages, of the same kind as each other compartment, but of a different skin tone; and

FIG. 14 is a top plan view of a non-print side of a blank used to form the carton of FIGS. 10-13 according to one embodiment of this invention.

### DETAILED DESCRIPTION OF THE INVENTION

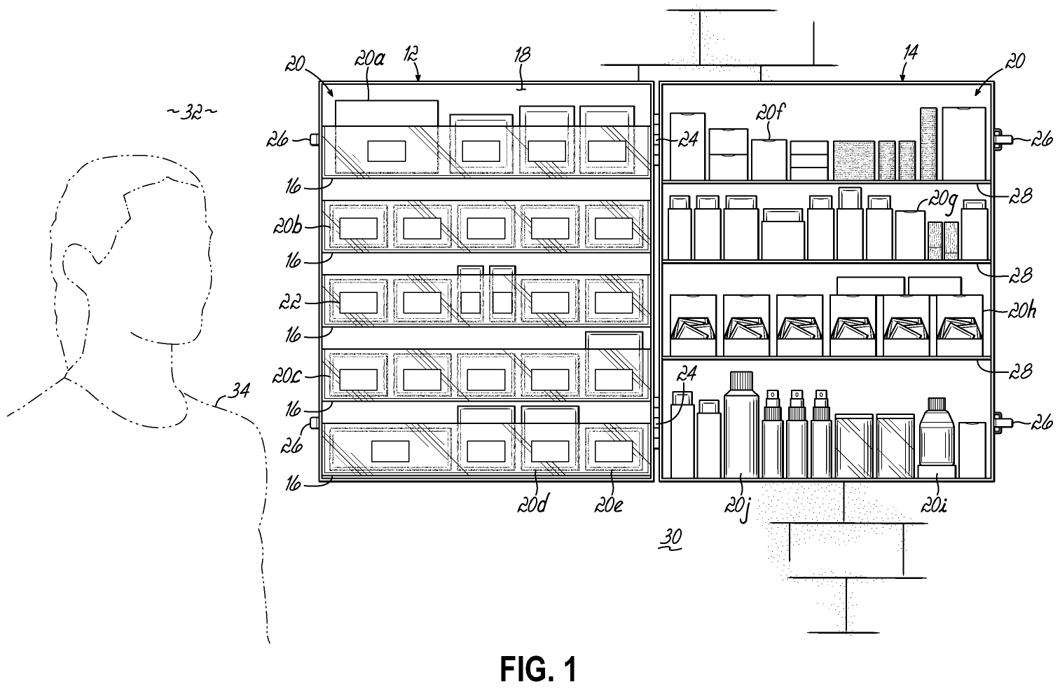

Referring to FIGS. 1-3, an exemplary embodiment of a first aid cabinet 10, a door 12 of the cabinet 10 and a housing portion 14 of the cabinet 10 according to one aspect of this invention is shown. It should be appreciated that the cabinet 10 and associated components shown in the drawings and/or described herein is but one of a variety of cabinets, lockers, cases, containers, depositories, closets, trunks, bags, packs, repositories and the like which are encompassed by this invention, any/all of which are referred to by the term cabinet herein for the sake of simplicity. The door 12 may have a number of pockets 16 on an inside face 18 thereof and each pocket 16 is adapted to hold and display any one of a variety of first aid items 20. Each pocket 16 may contain a plurality of a single type of first aid item 20 which may be readily identified by a label 22 on the leading such item 20 or container of such items 20 in a given pocket 16. The door 12 may be attached to the housing 14 by one or more hinges 24 to allow a user to selectively close and open the cabinet 10 as needed. A clasp 26 may be provided on the cabinet 10 to secure the door 12 in the closed position (not shown). One or more shelves 28 may be included in the housing 14 to support and display the first aid items 20. The cabinet 10 may be mounted on a wall 30 or other convenient location in a workplace 32 or another environment. A service sales representative (SSR) 34 as shown in FIG. 1 may periodically access the cabinet 10 for inspection, arranging, cleaning, maintenance, stocking and/or re-stocking the first aid items 20 contained therein.

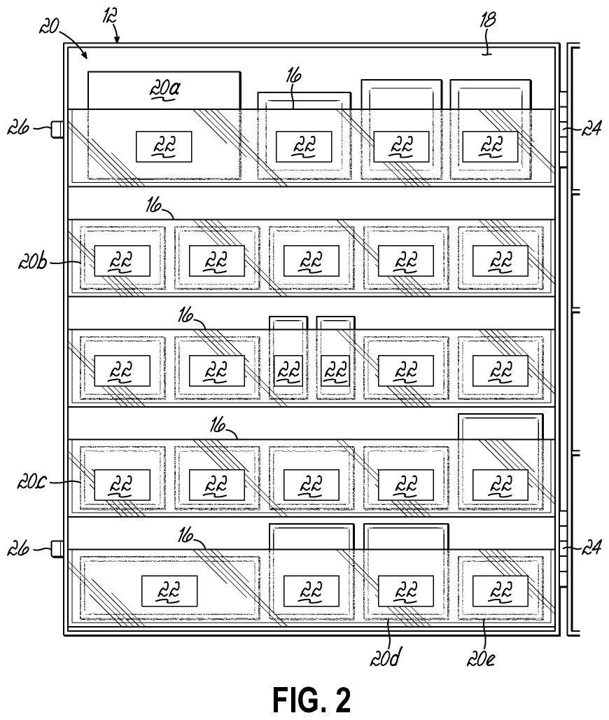

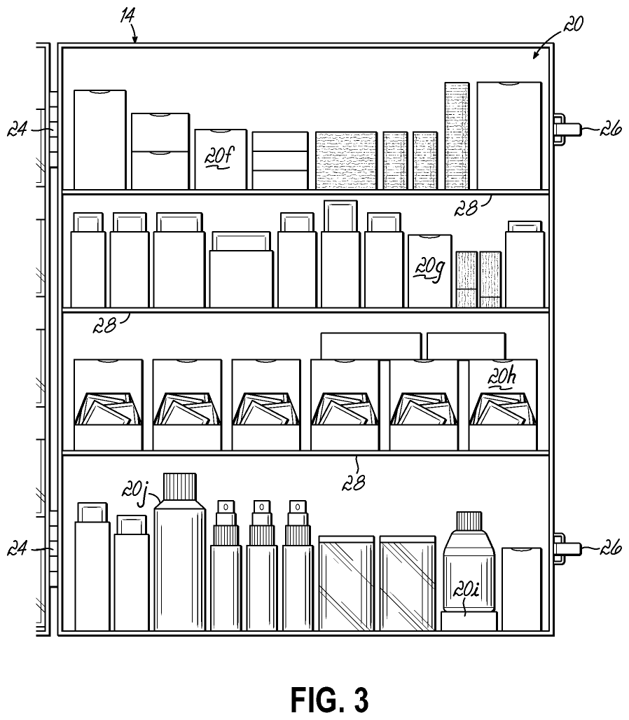

Referring to FIGS. 2-3, a variety of the first aid items 20 may be contained within the cabinet as desired, required and/or selected for the workplace environment serviced by the first aid cabinet 10. A first aid pocket guide 20a, individually packaged antiseptic wipes 20b, productivity aids 20c such as hand cleaners, diabetes control products 20d such as glucose tablets, and pain relievers 20e are examples of first aid items 20 which may be provided in the pockets 16 of the door 12 as shown in FIG. 2. Other exemplary first aid items 20 which may be provided on the shelves 28 of the housing include emergency response items 20f, bandages 20g, comfort tablets such as cough drops 20h, eye care items 20i such as eye wash and burn care items 20j such as ointment are shown in FIG. 3.

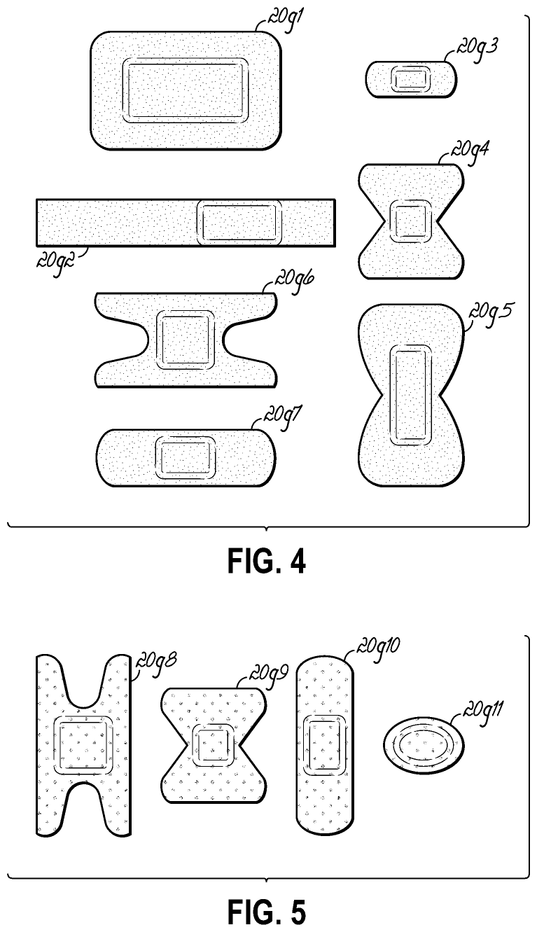

One aspect of various embodiments of this invention is the bandages 20g which may be provided in the cabinet 10. Examples of such bandages are shown in FIGS. 4-5 include large patch bandages 20g1, extra long strip bandages 20g2, junior size strip bandages 20g3, small knuckle bandages 20g4, large knuckle bandages 20g5, fingertip bandages 20g6, and elastic strip bandages 20g7 as in FIG. 4. Other types or shapes of bandages 20g may include comfort knuckle bandages 20g8, comfort fingertip bandages 20g9, comfort strip bandages 20g10 and comfort dot bandages 20g11 as in FIG. 5. These are but a few of the many types, shapes, sizes, and materials of bandages 20g which may be provided in the cabinet 10.

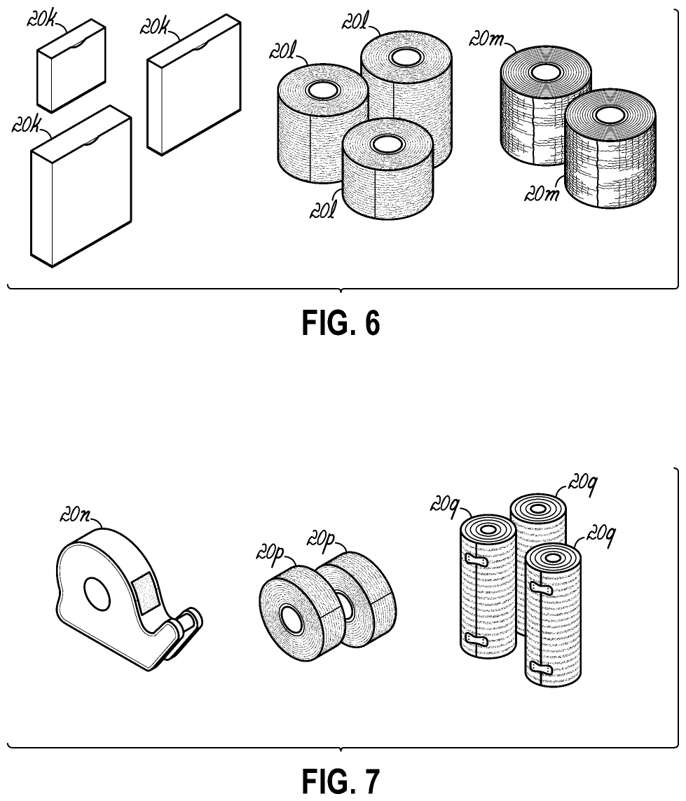

Other items 20 which may be included in the cabinet 10 include gauze pads 20k, rolls of wrap 201, and rolls of gauze 20m as shown in FIG. 6. Adhesive tape 20n, elastic tape 20p, and elastic wraps 20q are other examples of first aid items 20 as shown in FIG. 7.

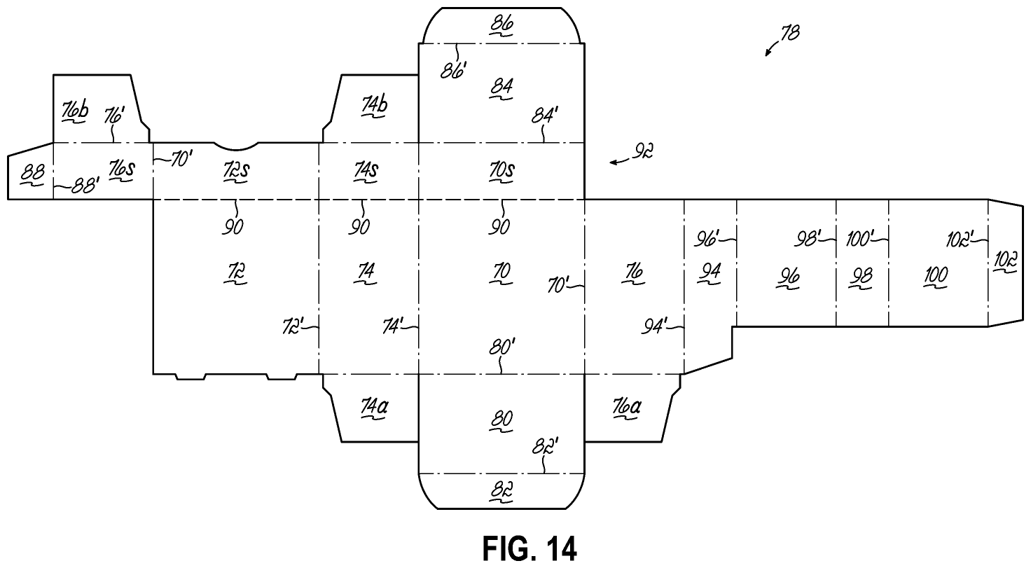

One aspect of many embodiments of this invention is that one or more first aid items 20 may be provided in a variety of skin tones or colors which may approximate or match the skin tone or color of a patient or user. One example of such items is an adhesive bandage 20g of a given size, shape, material and/or style which is provided in more than one color to match or approximate the skin tone of the patient or user as shown in FIG. 14. Multiple bandages 20g may be in a box, carton or container 40 as in FIG. 14 and the only difference between each of the bandages 20g in the box 40 is the color of the bandage 20g. Multiple bandages 20g′ of a first color are in a first compartment 42 of the box 40, multiple bandages 20g″ of a second color are in a second compartment 44 of the box 40 and multiple bandages 20g′″ of a third color are in a third compartment 46 of the box 40 according to the embodiment of this invention shown in FIG. 14. The first, second and third colors of the bandages 20g are different from each other and each may match or approximate the skin tone or color of a wearer according to one aspect of various embodiments of this invention. The bandages 20g of the first color are grouped together in the first compartment 42 and the bandages of the second and third color are each grouped together in the second and third compartments 44, 46, respectively, as shown in FIG. 14. While only three such bandages 20g are shown in each compartment 42, 44, 46, it will be appreciated that any number of items 20 may be included in each compartment. The box 40 in combination with the bandages 20g therein may be referred to as a package.

The bandages 20g according to one embodiment of this invention may have an individual wrapping 48 to maintain the item 20 clean and sterile until use.

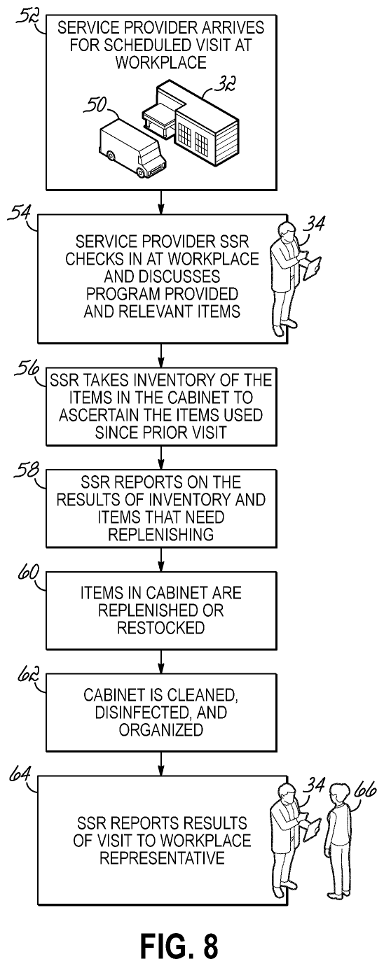

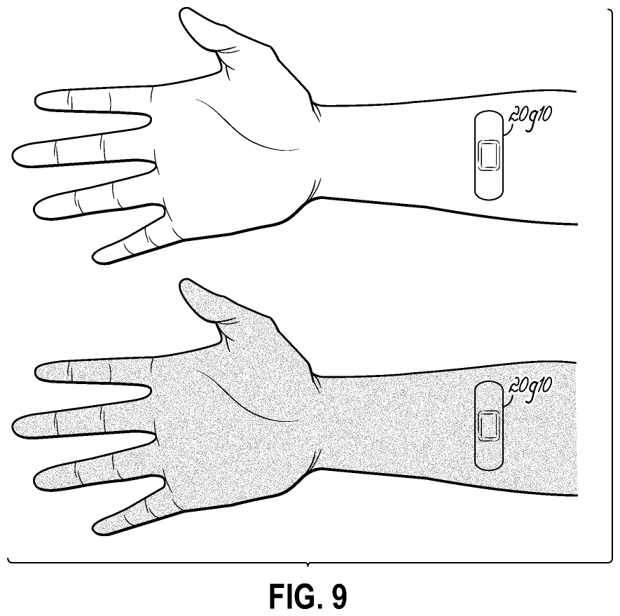

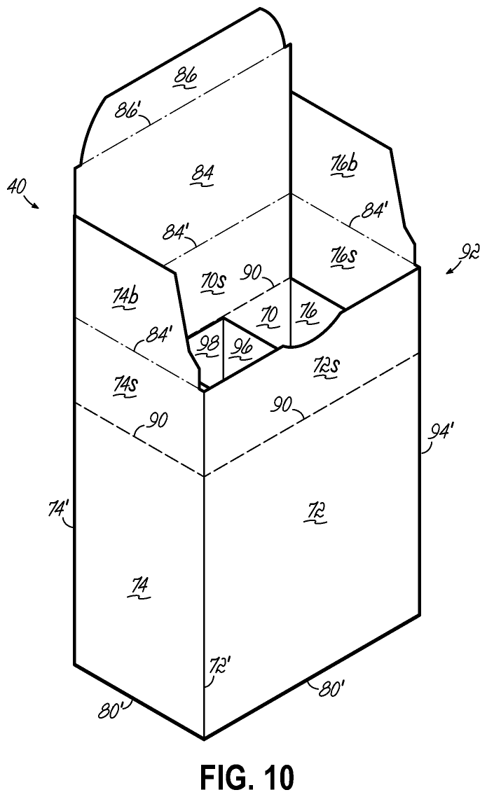

Referring to FIG. 8, one method for servicing the first aid cabinet 10 according to this invention is shown. Initially, a service provider 50 has a visit scheduled 52 at the workplace 32. The SSR 34 of the service provider 50 checks in 54 at the workplace 32 and discusses the program provided and the relevant items 20. A next step in one method is for the SSR 34 to take inventory 56 of the items 20 in the cabinet 10 to ascertain the items 20 used since the prior visit. The SSR 34 then reports 58 on the results of the inventory step 56. The items 20 in the cabinet 10 are then replenished or re-stocked 60. The cabinet 10 may then be cleaned and/or disinfected 62 as well as organized after the re-stocking step 60. The SSR 34 may then conclude the visit by reporting 64 the results of the visit a workplace representative 66 and discuss any additional needs related to the first aid cabinet 10 or other topics. As previously noted, the method of FIG. 8 is only one such method encompassed within this invention and variations thereof are included in this invention.

FIGS. 10-12 show one embodiment of the box 40 according to this invention and other box designs, sizes, configurations and shapes may be utilized within the scope of this invention. The box 40 includes a back panel 70 spaced from a front panel 72 by a pair of spaced side panels 74, 76. The front panel 72, side panel 74, back panel 70 and side panel 76 are serially joined together and separated by fold lines 72′, 74′, and 70′ as shown in a blank 78 shown in FIG. 15 to form the box 40. A bottom panel 80 of the box 40 is joined to the back panel 70 by a fold line 80′ extending to the side panels 74, 76. The fold line 80′ joins bottom side flaps 74a and 76a to the respective side panels 74, 76. A bottom tuck flap 82 is joined to the bottom panel 80 by a bottom tuck flap fold line 82′.

A top panel 84 of the box 40 is joined to the back panel 70 by a fold line 84′ extending to the side panel 74. The fold line 84′ joins top side flap 74b to the respective side panel 74. A top tuck flap 86 is joined to the top panel 84 by top tuck flap fold line 84′. A top side flap 76b is joined by fold line 76′ to the side panel 76 and a glue flap 88 is joined by fold line 88′ to a side free edge of side panel 76.

As seen in FIGS. 10, 12 and 15, upper portions 72s, 74s, 70s, and 76s of panels 72, 74, 70 and 76, respectively, are separable from the remainder of the associated panel 72, 74, 70, and 76 by a perforated tear line 90. The perforated tear line 90 defines a removable top portion 92 of the box as shown in FIG. 12 to expose the compartments 42, 44, 46 and items 20 therein as shown in FIG. 14.

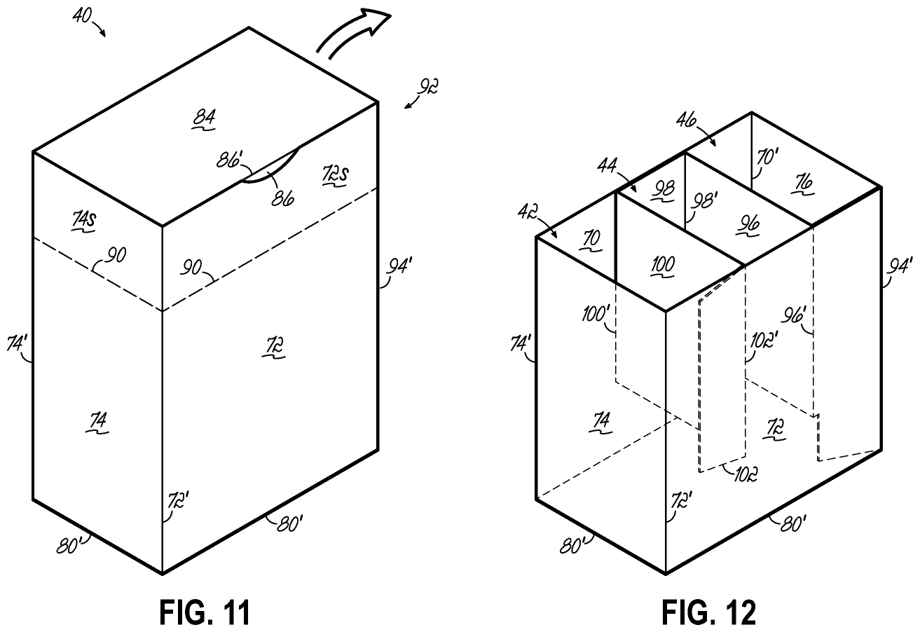

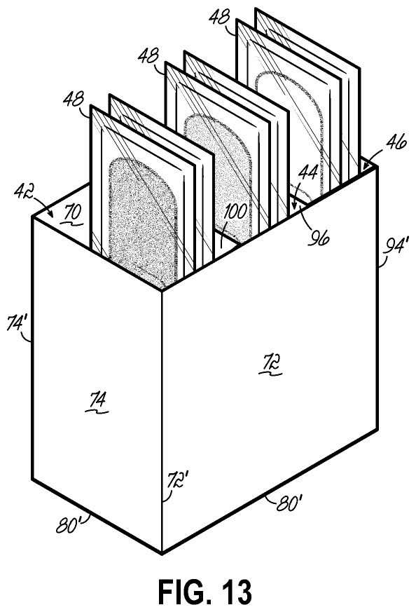

A glue flap 94 is joined to a side edge of the side panel 76 by a fold line 94′. A first divider panel 96 is joined to the glue flap 94 by a first divider panel fold line 96′. A spacer panel 98 is joined to the first divider panel 96 by a spacer panel fold line 98′. A second divider panel 100 is joined to the spacer panel 98 by a second divider fold line 100′. A glue flap 102 is joined to the second divider panel 100 by glue fold line 102′. As will be appreciated by one of ordinary skill in the art, the first and second divider panels 96, 100 help form the compartments 42, 44, 46 within the interior of the box 40 when the blank 10 is folded and glued into the erected box 40 as seen in FIGS. 11-13.

It should be appreciate by one of ordinary skill in the art that this invention is not limited to any particular number of compartments in the box 40 and three such compartments are shown in FIG. 14 as an example of but one embodiment of this invention. Additionally, items 20 other than bandages 20g may be contained in the compartments of the box 40 all of which may be similar with the exception of one feature, such as their color to match or approximate the skin tone of a user and, as a result, the compartment in which they are located in the box 40. Other examples of items 20 include, but are not limited to, wraps, pads, gauze, tape and other items 20. Moreover, the items 20 in the box 40 may be divided by color or skin tone as shown in FIG. 14 or another parameter or feature of the items 20 within the scope of this invention.

These foregoing embodiments of the invention are exemplary only and in no manner should be viewed as exhaustive or limiting on the scope of this invention. Alternative numbers of components, method steps, materials, parameters, arrangements and other aspects may be utilized within the scope of this invention.

From the above disclosure of the general principles of this invention and the preceding detailed description of at least one embodiment, those skilled in the art will readily comprehend the various modifications to which this invention is susceptible. Therefore, we desire to be limited only by the scope of the following claims and equivalents thereof.

We claim:

1. A package comprising:
a carton having a plurality of serially connected side panels, a top panel and a bottom panel;
at least one divider panel located within an interior volume of the carton, the at least one divider panel separating the interior volume of the carton into a first compartment and a second compartment;
a first plurality of first aid items located in the first compartment; and
a second plurality of first aid items located in the second compartment;
wherein all but a first one of a plurality of characteristics of the first plurality of first aid items are similar to the plurality of characteristics of the second plurality of first aid items.

2. The package of claim 1 wherein the first and second plurality of first aid items are both selected from the group of bandages, gauze pads, rolls of wrap, and rolls of gauze, adhesive tape, elastic tape, and elastic wrap.

3. The package of claim 1 wherein the first and second plurality of first aid items are both adhesive bandages.

4. The package of claim 1 wherein the first one of the plurality of characteristics is color.

5. The package of claim 4 wherein a first color of the first plurality of first aid items in the first compartment is different from a second color of the second plurality of first aid items in the second compartment.

6. The package of claim 5 wherein the first color approximates a first skin tone of a first user and the second color approximates a second skin tone of a second user.

7. The package of claim 1 wherein a first volume of the first compartment is equal to a second volume of the second compartment.

8. The package of claim 1 further comprising:
a first and a second divider panel both located within the interior volume of the carton, the first and the second divider panels separating the interior volume of the carton into the first compartment, the second compartment and a third compartment; and
a third plurality of first aid items located in the third compartment;
wherein all but the first one of the plurality of characteristics are similar among the first, second and third plurality of first aid items.

9. The package of claim 1 wherein the top panel is separable from the side panels to thereby provide access to the first and second plurality of first aid items in the first and second compartments, respectively.

10. The package of claim 1 wherein each of the first and second plurality of first aid items is individually wrapped.

11. The package of claim 1 further comprising:
a first replenish plurality of first aid items similar to the first plurality of first aid items and insertable into the first compartment to replenish a quantity of the first plurality of first aid items removed from the first compartment; and
a second replenish plurality of first aid items similar to the second plurality of first aid items and insertable into the second compartment to replenish a quantity of the second plurality of first aid items removed from the second compartment.

12. A package comprising:
a carton having a plurality of serially connected side panels, a top panel and a bottom panel;
at least one divider panel located within an interior volume of the carton, the at least one divider panel separating the interior volume of the carton into a first compartment and a second compartment;
a first plurality of first aid items located in the first compartment;
a second plurality of first aid items located in the second compartment;
wherein the first plurality of first aid items are similar to the second plurality of first aid items with the exception that the first plurality of first aid items are of a first color and the second plurality of first aid items are of a second and different color;
wherein the first color approximates a first skin tone of a first user and the second color approximates a second skin tone of a second user;
a first replenish plurality of first aid items similar to the first plurality of first aid items and insertable into the first compartment to replenish a quantity of the first plurality of first aid items removed from the first compartment; and
a second replenish plurality of first aid items similar to the second plurality of first aid items and insertable into the second compartment to replenish a quantity of the second plurality of first aid items removed from the second compartment.

13. The package of claim 12 wherein the first and second plurality of first aid items are both adhesive bandages each individually wrapped.

14. The package of claim 12 wherein the top panel is separable from the side panels to thereby provide access to the first and second plurality of first aid items in the first and second compartments, respectively.

15. A first aid cabinet located in a workplace and comprising:
a variety of first aid items contained within the first aid cabinet and accessible for retrieval from the first aid cabinet;
a package within the first aid cabinet, the package comprising
(a) a carton having a plurality of serially connected side panels, a top panel and a bottom panel;
(b) at least one divider panel located within an interior volume of the carton, the at least one divider panel separating the interior volume of the carton into a first compartment and a second compartment;
(c) a first plurality of first aid items located in the first compartment; and
(d) a second plurality of first aid items located in the second compartment;
wherein all but a first one of a plurality of characteristics of the first plurality of first aid items are similar to the plurality of characteristics of the second plurality of first aid items.

16. The first aid cabinet of claim 15 wherein the first and second plurality of first aid items are both adhesive bandages and the first one of the plurality of characteristics is color;
wherein a first color of the first plurality of first aid items in the first compartment is different from a second color of the second plurality of first aid items in the second compartment;
wherein the first color approximates a first skin tone of a first user and the second color approximates a second skin tone of a second user.

17. A method of servicing a first aid cabinet in a workplace, the method comprising the steps of:
putting a variety of first aid items in the first aid cabinet;
putting a package in the first aid cabinet, wherein the package comprises
(a) a carton having a plurality of serially connected side panels, a top panel and a bottom panel;
(b) at least one divider panel located within an interior volume of the carton, the at least one divider panel separating the interior volume of the carton into a first compartment and a second compartment;
inserting a first plurality of first aid items into the first compartment; and
inserting a second plurality of first aid items into the second compartment;
wherein all but a first one of a plurality of characteristics of the first plurality of first aid items are similar to the plurality of characteristics of the second plurality of first aid items.

18. The method of claim 17 wherein the first and second plurality of first aid items are both adhesive bandages and the first one of the plurality of characteristics is color;
the method further comprising the steps of:
selecting a first color of the first plurality of first aid items to approximate a first skin tone of a first user; and
selecting a second color to be different from the first color and to approximate a second skin tone of a second user.

19. The method of claim 17 further comprising:
assessing a quantity of the first plurality of first aid items in the first compartment;
adding to the quantity of the first plurality of first aid items in the first compartment;
assessing a quantity of the second plurality of first aid items in the second compartment; and
adding to the quantity of the second plurality of first aid items in the second compartment.

20. The method of claim 17 further comprising:
removing the top panel from the carton to allow for access to the first and second plurality of first aid items in the first and second compartments, respectively.
